(19) United States
(12) Patent Application Publication
(10) Pub. No.: US 2006/0127483 A1
(43) Pub. Date: Jun. 15, 2006

(54) Topical drug delivery system

(76) Inventors: Alan Drizen, Toronto (CA); John G. Guerra, Burlington (CA)

Correspondence Address: MORGAN & FINNEGAN, L.L.P., 3 World Financial Center, New York, NY, 10281-2101, US

(21) Appl. No.: 11/010,154

(22) Filed: Dec. 10, 2004

(51) Int. Cl. A61K 9/14

(52) U.S. Cl. 424/488

(57) ABSTRACT

The present invention relates to topical drug compositions and methods for topical drug delivery which promote stability of a drug component and facilitate the penetration of the drug component into the skin of the host. The invention also relates to topical drug compositions containing a suitable vasoactive agent, such as a prostaglandin, and methods for effectively delivering said active ingredient to the host. These compositions and methods are useful for the treatment of sexual dysfunction, in both men and women. The invention also relates to methods for formulating or preparing the composition of the present invention. To minimize irritation, the composition is water based.

### BACKGROUND OF THE INVENTION

1. Field of the Invention

The present invention relates to topical drug compositions and methods for topical delivery of drugs. Of particular relevance are drug delivery systems for the treatment of sexual dysfunction.

2. Background of the Invention

Sexual dysfunction in men and women has been the subject of extensive investigation and review in recent years. The American Foundation for Urologic Disease (AFUD) has developed a classification system to be used by professionals treating female sexual dysfunction. Under this system, female sexual dysfunction includes: hypoactive sexual desire disorder, sexual aversion disorder, sexual arousal disorder, orgasmic disorder and sexual pain disorders. The cause of female sexual dysfunction is not well understood but likely involves vascular, neurological, hormonal and psychogenic factors. Any condition that results in reduced blood flow to the vagina or clitoris can result in sexual dysfunction. Engorgement of these tissues with blood is necessary for producing sexual sensations and for the production of the necessary lubrication.

Neurological disorders can also cause reduced sensation in the vagina and clitoris resulting in sexual dysfunction. As women approach menopause, changes in hormone levels, particularly estrogen and testosterone, can lead to changes in sexual function. Estrogen is needed to help maintain adequate blood flow, muscle tone and lubrication as well as an adequate level of desire. While levels of testosterone are much lower in females than males, adequate levels still are needed in the female to maintain normal desire and libido. Psychological conditions such as anxiety, depression and obsessive compulsive disorder can all be associated with female sexual dysfunction. In addition, some medications used to treat these conditions, such as serotonin re-uptake inhibitors, are associated with reduced desire and libido in women.

In men, sexual dysfunction is usually referred to as erectile dysfunction. Erectile dysfunction is defined as the repeated inability to maintain an erection sufficient for satisfactory intercourse. The are a number of potential causes of erectile dysfunction that involve vascular, neurological, hormonal and psychogenic factors. Vascular function is particularly important since an erection is the result of the penis becoming engorged with blood. In addition to organic causes, erectile dysfunction can be the result of certain medications, in particular certain medications used to treat hypertension.

Treatment options for sexual dysfunction are currently more limited for women than for men. In women, estrogen replacement therapy can relieve many of the symptoms of sexual dysfunction such as vaginal dryness as well as reduce or eliminate symptoms associated with menopause. However, recent studies showing an increase in cardiovascular disease and stroke in women with hormone replacement therapy have raised concerns about the safety of this approach. Treatment with testosterone can increase libido but can also result in weight gain and increased facial hair.

Several orally administered drug products have been developed for treatment of men's erectile dysfunction, such as VIAGRA® (sildenafil citrate), LEVITRA® (vardenafil HCl) and CIALIS® (tadalafil). These phosphodiesterase inhibitors are widely used to treat male sexual dysfunction but trials of these drugs in women with sexual dysfunction have generally had disappointing results.

Prostaglandin E1 (Alprostadil) has been used in the treatment of sexual dysfunction in both men and women. Prostaglandin E1 is administered locally to function as a smooth muscle relaxant and a vasodilator. In the penis and clitoris, prostaglandin E1 acts to relax the trabecular smooth muscle and to dilate the arteries. The significant disadvantage of products containing prostaglandin E1 is the method of their administration. The products for men containing prostaglandin E1 are CAVERJECT® by Pfizer and MUSE® by Vivus Inc. In the case of CAVERJECT®, the prostaglandin E1 is injected directly into the penis. While generally effective, the method for administering CAVERJECT® is painful and unpleasant. MUSE® is a small suppository containing Prostaglandin E1 that is inserted into a man's urethra. Again, the method of administration is unpleasant.

Vivus Inc. is developing the product ALISTA® containing prostaglandin E1 for the treatment of female sexual dysfunction. U.S. Pat. Nos. 5,877,216, 6,593,313 and 6,593,369, assigned to Vivus Inc., relate to use of prostaglandin E1 in the treatment of female dysfunction, topical application of prostaglandins, optionally including the addition of an androgen, application to the clitoris and/or vulva of a female suffering from sexual dysfunction.

The product FEMPROX developed by NexMed Inc. of Robbinsville, N.J. contains prostaglandin E1 as the primary active ingredient and is currently in clinical trials for the treatment of female sexual dysfunction. U.S. Pat. No. 6,486,207 assigned to NexMed Inc. describes compositions containing prostaglandin E1 and N,N-disubstituted amino alkonates which act as penetration enhancers for the prostaglandin.

### SUMMARY OF THE INVENTION

The present invention relates to topical drug compositions and methods for topical drug delivery which promote stability of a drug component and facilitate the penetration of the drug component into the skin of the host. The invention also relates to topical drug compositions containing a suitable vasoactive agent, such as a prostaglandin, and methods for effectively delivering said active ingredient to the host. These compositions and methods are useful for the treatment of sexual dysfunction, in both men and women. The invention also relates to methods for formulating or preparing the composition of the present invention. To minimize irritation, the composition is water based.

The topically applied formulations of the present invention incorporate a drug and deliver pharmacologically effective amounts of the drug to a targeted area of the host or patient. The topical drug composition of the present invention is an aqueous formulation that includes at least one vasoactive agent, one or more preservatives, solvents, humectants, viscosity increasing agents and emulsifying agents.

Vasoactive agents are agents that affect either the constriction or relaxation of blood vessels. Suitable vasoactive agents include, but are not limited to, prostaglandins in particular prostaglandin E1. In some embodiments of the invention, prostaglandins are from about 0.05% to about 0.15% by weight of the composition.

Suitable preservatives include, but are not limited to, alkyl hydroxybenzoates. Solvents include but are not limited to alkanols, in particular C₁-C₆ alkanols. Humectants include but are not limited to alkoxylated alcohols. Viscosity increasing agents include but are not limited to hydrophilic colloids, in particular high molecular weight colloids. Emulsifying agents include but are not limited to fatty acid esters.

In one embodiment of the invention, the composition includes the vasoactive agent and an aqueous solution of p-hydroxybenzoic acid methyl ester, ethanol, methoxypolyethylene glycol, hydroxyethylcellulose and carboxymethylcellulose and optionally one or more of propylene glycol, polyvinyl alcohol and polysorbate. In a further embodiment of the invention the composition includes, in addition to the vasoactive agent, the percent by weight: p-hydroxybenzoic acid methyl ester from about 0.1% to about 0.5%; ethanol from about 6% to about 10%; methoxypolyethylene glycol from about 5% to about 20%; propylene glycol from about 2% to about 4%; hydroxyethylcellulose from about 0.1% to about 1%; carboxymethylcellulose from about 0.1% to about 1%; and naturally occurring or synthetic prostaglandin from about 0.05% to about 0.5%.

In a further embodiment of the invention the aqueous solution includes p-hydroxybenzoic acid methyl ester, ethanol, methoxypolyethylene glycol, hydroxyethylcellulose and carboxymethylcellulose and propylene glycol. In a still further embodiment of the invention from about 0.1% to about 0.5% p-hydroxybenzoic acid methyl ester; from about 6% to about 10% ethanol; from about 5% to about 10% methoxypolyethylene glycol; from about 2% to about 4% propylene glycol; from about 0.1% to about 1% hydroxyethylcellulose; from about 0.1% to about 1% carboxymethylcellulose; and from about 0.05% to about 0.5% naturally occurring or synthetic prostaglandin all in percent by weight are present.

In a further embodiment of the invention the composition includes an aqueous solution of p-hydroxybenzoic acid methyl ester, ethanol, methoxypolyethylene glycol, hydroxyethylcellulose and carboxymethylcellulose, polyvinyl alcohol and polysorbate. In a preferred embodiment of the invention the composition comprises, the percent by weight: p-hydroxybenzoic acid methyl ester from about 0.1% to about 0.5%; ethanol from about 6% to about 10%; methoxypolyethylene glycol from about 10% to about 25%; polyvinyl alcohol from about 0.5% to about 3%; polysorbate from about 2% to about 8%; hydroxyethylcellulose from about 0.1% to about 1%; carboxymethylcellulose from about 0.1% to about 1%; and naturally occurring or synthetic prostaglandin from about 0.05% to about 0.5%.

In another embodiment, the prostaglandin component present in the composition is substantially entirely prostaglandin E1. In a further embodiment, the prostaglandins other than prostaglandin E1 comprise less than about 1.5% of the total prostaglandins.

The invention also includes a method for treating sexual dysfunction by topically applying the above described composition to a desired region of the skin of the host. In a particular embodiment, a female desiring treatment for sexual dysfunction applies said composition to the vagina and/or clitoral regions. The composition is preferably applied about ten minutes prior to sexual activity.

Another embodiment of the invention is a method for treating erectile dysfunction in males by applying to the skin of the penis the above described composition to deliver the vasoactive agent, such as a prostaglandin, directly to the penis. In a preferred embodiment the composition is applied to the penis within about ten minutes prior to sexual activity. In a further embodiment, when the composition is intended for use by males, the number and concentration of viscosity increasing agents and emulsifying agents is increased.

### DETAILED DESCRIPTION OF THE INVENTION

The invention involves a process of formulating the composition described above. The method of preparation comprises the sequential addition of the following ingredients to water at one or more elevated temperatures in the range of about 55° C. to about 90° C. during stirring said ingredients comprising p-hydroxybenzoic acid methylester, hydroxyethylcellulose, carboxymethylcellulose and methoxypolyethylene glycol; and when the composition is at a temperature below about 60° C. adding a solution of a prostaglandin and ethanol. In a further embodiment of the process, the process comprises heating water to about 90° C. and adding p-hydroxybenzoic acid methyl ester to the water at the heated temperature. After the addition the water is allowed to cool while stirring at about 450 rpm. When the temperature of the aqueous composition is about 75° C. to about 80° C., hydroxyethylcellulose and carboxymethylcellulose are added and stirring of the aqueous composition at about 450 rpm is continued. When the temperature of the water is about 55° C. to about 60° C. methoxypolyethylene glycol is added and stirring is continued at 450 rpm. A naturally occurring or synthetic prostaglandin is dissolved in ethanol and the resulting ethanol solution is added to the aqueous composition with stirring at about 300 rpm. While stirring, propylene glycol is added to the aqueous composition. After addition of the propylene glycol stirring is reduced to a speed of about 90 rpm. In one embodiment, the composition is packaged in a suitable container. In a further embodiment a single dosage amount of the composition is packaged in a disposable container.

One of the components of the composition is an alkyl hydroxybenzoate preservative such as p-hydroxybenzoic acid methyl ester, also known as methyl 4-hydroxybenzoate and as methyl paraben. In the present invention, the free base form of methyl paraben is preferably used. Methyl paraben (free base) is readily available in grades suitable for use in pharmaceutical and cosmetic compositions from a variety of commercial suppliers.

An alkanol solvent, such as ethanol, is another ingredient in the composition of the invention and is preferably absolute ethanol (free of water), which is also readily available from commercial sources. Other C₁-C₆ lower alkanols including methanol, butanol, propanol, pentanol and hexanol are also suitable for use in the composition.

Alkoxylated alcohol humectants for use in the invention include methoxypolyethylene glycol, methoxypolypropylene glycol and methoxypolyvinyl glycol. The alkoxylated alcohol preferably has an average molecular weight of from about 300 to about 750 daltons, preferably about 350 daltons. These materials are readily available from commercial sources.

The composition of the present invention also contains one or more hydrophilic colloids to act as viscosity increasing agents. Suitable hydrophilic colloids are of high molecular weight and include carboxymethylcellulose and hydroxyethylcellulose. Suitable carboxymethylcelluloses that can be used in the invention have a viscosity from about 2,000 cps to about 5,000 cps in a 1% solution. Preferred hydroxyethylcelluloses suitable for use in the invention have a viscosity from about 1,000 cps to about 3,000 cps in a 1% solution.

The hydrophilic colloid component has a high average molecular weight, e.g. in the order of 10,000-15,000 daltons. Suitable materials are available from Hercules Inc. of Wilmington Del. For example the carboxymethylcellulose product AQUALON® CMC 7H3SXF and the hydroxyethylcellulose product NATROSOL® 250H NF grade are suitable materials.

The composition of the present invention may also contain one or more emulsifying agents which can be fatty acid esters such as polysorbate or polyols such as polyvinyl alcohol. The fatty acid esters preferably have a molecular weight greater than 1,000 daltons.

The preferred vasoactive agent is a prostaglandin, most preferably prostaglandin E1, which has the generic drug name of Alprostadil. It is preferred that when present the prostaglandin E1 be highly pure. In a further preferred embodiment, when prostaglandin E1 is present, prostaglandins other than prostaglandin E1 comprise less than 1.5% of the total prostaglandins in the composition. A suitable prostaglandin E1 is available from Chinoin Pharmaceutical and Chemical Works Co. Ltd. of Budapest, Hungary.

For best results the composition components are combined in accordance with the following protocol. As a result prostaglandin E1 potency is preserved and penetrability through the skin enhanced. Table 1 describes the components in a composition of the invention including preferred ranges for the concentrations of the components in the composition.

TABLE 1

| Compound | W/W % |
|---|---|
| Methyl Paraben | 0.1%-0.5% |
| Absolute Ethanol | 6%-10% |
| Methoxypolyethylene Glycol | 5%-20% |
| Hydroxyethylcellulose HW | 0.1%-2% |
| Carboxymethylcellulose HW | 0.1%-2% |
| Propylene Glycol (optional) | 2.5%-5.0% |
| Polyvinyl Alcohol (optional) | 0.1%-2% |
| Polysorbate 80 (optional) | 2%-5% |
| Purified Water USP | balance |

USP grade water is heated to about 90° C. and the heat is turned off. Methyl paraben (free base) is added to the heated water for 2 hours while stirring at about 450 rpm. Hydroxyethylcellulose and carboxymethylcellulose are mixed together in equal proportions by weight. When the temperature of the aqueous composition is about 70-80° C., an equal proportional mixture of hydroxyethylcellulose and carboxymethylcellulose is added while stirring at 450 rpm for about 30-35 minutes. When the temperature of the aqueous composition cools to about 55-60° C., methoxypolyethylene glycol is added while stirring at about 450 rpm for 6 hours. In a separate stirrer, the prostaglandin E1 is dissolved in absolute ethanol while stirring at slow speed for 5 minutes. The prostaglandin E1 ethanol solution is added to the aqueous composition while stirring at about 300 rpm for 1 hour. Propylene glycol is added to the aqueous composition while stirring at about 300 rpm for 1 hour. After the 1 hour, the stirring speed is reduced to about 90 rpm and the stirring of the aqueous composition continues overnight. The completed composition is packaged in suitable containers. It has been found that tubes made of polypropylene provide optimal stability for the prostaglandin E1. To maintain stability of the prostaglandin E1 during long term storage, the composition is stored at −15° C. to −20° C. On thawing, the composition is stable at room temperature for up to 30 days although the composition can be refrozen if storage for a longer period of time is desired.

For the stirring steps of the above procedure, stirrers that are capable of maintaining a constant stirring speed even when the viscosity of the solution is increased are preferred. Such stirrers are referred to as fixed torque stirrers. Suitable stirrers include model BDC303 produced by Caframo Limited of Wiarton, Canada.

The composition of the present invention is a viscous aqueous gel-like solution. The gel like nature of the composition is maintained when the composition is frozen and thawed. When used for the treatment of female sexual dysfunction the composition is preferably applied to the perineal area of the female host, in particular the vagina and clitoris. This allows the prostaglandin to increase the blood flow to these tissues resulting in the increased production of lubrication and increased tactile sensitivity, thereby improving the sexual function of the host. A single dose of the composition is about 2-3 ml and should be applied shortly before sexual activity, preferably not more than an hour before sexual activity. The effect of the prostaglandin lasts for about one hour. After the one hour period the composition may be reapplied if necessary.

In the case of the treatment of erectile dysfunction, the composition is preferably applied directly to the penis so that blood flow can be increased leading to an improved erection. The usual dose is preferably about 2-3 ml and is applied shortly before sexual activity, preferably not more than ten minutes before sexual activity.

Improvement in sexual function can be evaluated by the use of questionnaires or diaries where the patient records the quality of their sexual experiences. Data collection should include a pretreatment baseline period of 4-8 weeks. Specific endpoints of the sexual experience include satisfactory sexual intercourse, sexual intercourse resulting in orgasm, oral sex resulting in orgasm and partner-initiated or self masturbation resulting in orgasm. A statistically significant change in the frequency of successful and satisfactory sexual experiences over time provides a measure of the effectiveness of the treatment for sexual function. The determination of what is a successful and satisfactory sexual experience is made by the patient not the patients partner. Physical changes such as increased blood flow to the genitals, engorgement of the penis or clitoris or changes in vaginal lubrication can also be measured but should be considered as secondary supportive information and not a primary indication of the success or failure of the treatment.

Questionnaires which can be used to evaluate sexual function include the Female Sexual Distress Scale described by Derogatis et. al. (J. Sex Marital Ther. 28(4):317-30, 2002) and the Female Sexual Function Index described by Rosen et. al. (J. Sex Marital Ther. 26(2): 191-208, 2000). And for males the International Index of Erectile Function (IIEF) described by Rosen et. al. (Int. J. Impotence Res. 11:319-326, 1999) can be used.

### EXAMPLES

### Example 1

A number of formulations are shown in Table 2 below to illustrate the effect of changes in components on the stability of the prostaglandin active ingredient and aesthetic feel and appearance of the formulation.

TABLE 2

| | A | B | C | D | E | F |
|---|---|---|---|---|---|---|
| USP Water | 82.475 | 72.375 | 78.275 | 82.32 | 80.72 | 80.72 |
| Methyl Paraben | 0.2 | | | | 0.2 | 0.2 |
| Na Hyaluronate | 0.7 | 1.5 | 1.1 | | | |
| Alprostadil | 0.125 | 0.125 | 0.125 | 0.08 | 0.08 | 0.08 |
| Ethanol | 10 | 7 | 5 | 7 | 8 | 8 |
| Methoxypolyethylene | | 5 | 10 | 7 | 7 | 7 |
| glycol 350 | | | | | | |
| Propylene glycol | | 5 | 2.5 | 3 | 3 | 3 |
| Hydroxyethyl- | | | | | | 0.5 |
| cellulose HW | | | | | | |
| Hydroxyethyl- | | | | | 0.5 | |
| cellulose MW | | | | | | |
| Hydroxyethyl- | | | | 0.3 | | |
| cellulose LW | | | | | | |
| Carboxymethyl- | | | 0.5 | | | 0.5 |
| cellulose HW | | | | | | |
| Carboxymethyl- | | | | 0.3 | 0.5 | |
| cellulose MW | | | | | | |
| Carboxymethyl- | 0.5 | 1 | | | | |
| cellulose LW | | | | | | |
| Polyethylene Glycol | 3 | 5 | 2.5 | | | |
| Glycerin | 3 | 3 | | | | |

Batch A was prepared in 4 stages with the Alprostadil added in the third stage and the glycerin added last. This batch turned cloudy and had crystal formation after two hours.

Batch B was prepared in 4 stages with the Alprostadil added in the second stage followed by the hyaluronate and glycerin. This batch turned cloudy when the hyaluronate was added and required additional ethanol to clarify the solution.

Batch C contained carboxymethylcellulose HW as an additional component and the order of adding the components was changed whereby the Alprostadil was added in the first stage followed by water, carboxymethylcellulose HW and hyaluronate. This batch also turned cloudy.

Batch D was prepared in two stages with Alprostadil added in the second stage. Hyaluronate was suspected of reacting with other components of the formulation and was not used in this batch. The combination of carboxymethylcellulose and hydroxyethylcellulose improved the stability of the formulation however, the viscosity of the formulation was very low.

Batch E was prepared in three stages with the methyl paraben added first and the Alprostadil last. The concentrations of the carboxycelluloses were increased to 0.5% and medium weight versions were used for both. The viscosity improved but was still low.

This led to batch F where the high weight versions of both carboxymethylcellulose and hydroxyethylcellulose were used. This batch was prepared in four stages with the methyl paraben added in the first stage and the Alprostadil dissolved in the ethanol and added in the fourth stage. This batch remained clear and had good viscosity characteristics.

The viscosity and feel of the composition of Batch F are suitable for use in applying a drug to the mucous membranes found in the female perineum and vagina. However, Batch F would not be as well-suited for application to the skin that is found on the male penis. The skin of the penis contains a stratum corneum which is keratinized and less permeable than the mucous membrane of the female. Also since the penis is not an enclosed cavity the composition needs to be more viscous so that it remains in place on the skin and does not run off.

### Example 2

To illustrate the importance of carefully choosing ingredients and the particular method of preparation, various formulations are described below.

TABLE 3

| | A | B | C | D | E | F | G |
|---|---|---|---|---|---|---|---|
| USP Water | 78.3 | 80.7 | 65.7 | 74.7 | 69.7 | 71.7 | 64.3 |
| Methyl Paraben | 0.2 | 0.2 | 0.2 | 0.2 | 0.2 | 0.2 | 0.2 |
| Na Hyaluronate | 0.9 | 0.5 | | | | | |
| Alprostadil | 0.08 | 0.08 | 0.08 | 0.08 | 0.08 | 0.08 | 0.08 |
| Ethanol | 10 | 10 | 10 | 7 | 7 | 7 | 7 |
| Methoxypolyethylene | | | 15 | 15 | 15 | 15 | 20 |
| glycol 350 | | | | | | | |
| Hydroxyethyl- | | | | | | | 1 |
| cellulose HW | | | | | | | |
| Hydroxyethyl- | | | | | | 0.5 | |
| cellulose LW | | | | | | | |
| Carboxymethyl- | | | | | 1 | 0.5 | 1 |
| cellulose HW | | | | | | | |
| Carboxymethyl- | 0.5 | 0.5 | 1 | 1 | | | |
| cellulose LW | | | | | | | |
| Polyvinyl Alcohol | | | | | | | 1.4 |
| Polysorbate 80 | | | | | 5 | 5 | 5 |
| Polyethylene Glycol | 5 | 4 | 5 | 2 | 2 | | |
| Glycerin | 5 | 4 | 3 | | | | |

Batch A was prepared in three stages with the methyl paraben and Hyaluronate being added in the first stage and the ethanol and Alprostadil added at the end. In this formulation the mixture became cloudy when the Alprostadil was added.

In batch B, the concentrations of hyaluronate, polyethylene glycol and glycerin were reduced and the glycerin was not added until the end of the formulation process. This batch also became cloudy after all of the components had been added.

In batch C hyaluronate was not used and in it's place was present methoxypolyethylene glycol 350. This batch was clear after mixing but became cloudy when stored in the refrigerator.

Batch D differed from the foregoing batches by the absence of glycerin as a component and the reduction of the concentrations of polyethylene glycol and ethanol. This batch was clear after mixing and remained clear after refrigeration.

For Batch E, polysorbate 80 was included as a new component and the carboxymethylcellulose used had a higher average molecular weight than the carboxymethylcellulose components of the above-described batches. While this batch remained clear, on standing, fine lines appeared in the formulation.

In Batch F, hydroxyethylcellulose was used as a new component and, as was found with the formulations in example 1, the combination of carboxymethylcellulose and hydroxyethylcellulose greatly improved the stability of the composition.

In Batch G, polyvinyl alcohol was used as a new component and the concentrations of the cellulose components were increased. This resulted in a composition with improved viscosity that would be well-suited for application to the skin of the penis.

### Example 3

This example illustrates a study of the effectiveness of the delivery of a topical drug composition of the present invention containing Alprostadil as the active drug ingredient. The topical absorption of Alprostadil in human skin was studied in vitro using tritium labeled Alprostadil. Dermatomed human female abdominal skin, obtained for other reasons from two separate donors, was used in the study. The skin was tape-stripped 10 times to remove the stratum corneum so that it would better mimic the behavior of labial skin. The skin was mounted on Franz static-type diffusion cells maintained at a constant temperature of 32° C. Five diffusion cells were used for each of the two donors. The Alprostadil formulation of Batch F of Table 2 was applied to the skin for 20 minutes and then excess material removed with cotton swabs and a single cellophane tape-strip. The dermis and epidermis were separated and analyzed separately. The following table gives the amount of the applied dose that reaches each skin layer or completely penetrates the skin (receptor) for each of the two donors.

TABLE 3

| Tissue | % of Applied Dose |
|---|---|
| Epidermis | 9.4 ± 0.8 |
| | 5.6 ± 1.5 |
| Dermis | 0.23 ± 0.24 |
| | 0.26 ± 0.15 |
| Receptor | 0.004 ± 0.001 |
| | 0.026 ± 0.021 |

The results in Table 3 show that by 20 minutes, Alprostadil had already penetrated the epidermis, entered the dermis and started to enter the receptor solution on the other side of the skin. This indicates that not only does the formulation of batch F have good stability and viscosity characteristics but that it also allows the topical absorption of Alprostadil into and through the skin.

What is claimed is:

1. A composition for topical delivery to a host comprising an aqueous formulation of one or more vasoactive agents, one or more of a alkyl hydroxybenzoate preservative, a alkanol solvent, a alkoxylated alcohol humectant, and a high molecular weight hydrophilic colloid viscosity increasing agent.

2. The composition of claim 1 further comprising one or more emulsifying agents.

3. The composition of claim 1 wherein:
(a) the alkyl hydroxybenzoate preservative is methyl paraben which is from about 0.1% to about 0.5% by weight of the composition,
(b) the alkanol solvent is ethanol which is from about 6% to about 10% by weight of the composition,
(c) the alkoxylated alcohol humectant is methoxypolyethylene glycol which is from about 5% to about 25% by weight of the composition,
(d) the high molecular weight hydrophilic colloid viscosity increasing agent is selected from one or more of the group consisting of carboxymethylcellulose and hydroxyethylcellulose and which is from about 0.1% to about 2% by weight of the composition.

4. The composition of claim 1 wherein:
(a) the alkyl hydroxybenzoate preservative is methyl paraben which is from about 0.1% to about 0.5% by weight of the composition,
(b) the alkanol solvent is ethanol which is from about 6% to about 10% by weight of the composition,
(c) the alkoxylated alcohol humectant is methoxypolyethylene glycol which is from about 5% to about 10% by weight of the composition,
(d) the high molecular weight hydrophilic colloid viscosity increasing agent is selected from one or more of the group consisting of carboxymethylcellulose and hydroxyethylcellulose and which is from about 0.1% to about 2% by weight of the composition.

5. The composition of claim 2 wherein:
(a) the alkyl hydroxybenzoate preservative is methyl paraben which is from about 0.1% to about 0.5% by weight of the composition,
(b) the alkanol solvent is ethanol which is from about 6% to about 10% by weight of the composition,
(c) the alkoxylated alcohol humectant is methoxypolyethylene glycol which is from about 10% to about 25% by weight of the composition,
(d) the high molecular weight hydrophilic colloid viscosity increasing agent is selected from one or more of the group consisting of carboxymethylcellulose and hydroxyethylcellulose and which is from about 0.1% to about 2% by weight of the composition,
(e) the fatty acid ester emulsifying agent is polysorbate 80 which is from about 2% to about 8% by weight of the composition.

6. The composition of claim 1 wherein the composition is an aqueous gel material.

7. The composition of claim 1 wherein the vasoactive agent is a naturally occurring or synthetic prostaglandin.

8. The composition of claim 6 wherein the naturally occurring or synthetic prostaglandin is naturally occurring or synthetic prostaglandin E1.

9. The composition of claim 8 wherein the naturally occurring or synthetic prostaglandin E1 is from about 0.05% to about 0.5% percent by weight.

10. The composition of claim 9 wherein prostaglandins other than the prostaglandin E1 comprise less than about 1.5% by weight of the total prostaglandins.

11. A method for treating sexual dysfunction in a person comprising applying to the genital organs an aqueous formulation comprising one or more of a vasoactive agent, a alkyl hydroxybenzoate preservative, a alkanol solvent, a alkoxylated alcohol emulsifying agent, and a high molecular weight hydrophilic colloid viscosity increasing agent to a region of the body.

12. The method of claim 11 wherein:
(a) the alkyl hydroxybenzoate preservative is methyl paraben which is from about 0.1% to about 0.5% by weight of the composition,
(b) the alkanol solvent is ethanol which is from about 6% to about 10% by weight of the composition,
(c) the alkoxylated alcohol humectant is methoxypolyethylene glycol which is from about 5% to about 25% by weight of the composition,
(d) the high molecular weight hydrophilic colloid viscosity increasing agent is selected from one or more of the group consisting of carboxymethylcellulose and hydroxyethylcellulose and which is from about 0.1% to about 2% by weight of the composition.

13. The method of claim 12 wherein the person is a female and the composition is applied to the vagina.

14. The method of claim 12 wherein the person is a female and the composition is applied to the clitoris.

15. The method of claim 12 wherein the person is a female and the composition is applied to the vagina and the clitoris.

16. The method of claim 12 wherein the person is a male and the composition is applied to the penis.

17. The method of claim 12 wherein the composition is applied no more than about ten minutes before sexual activity.

18. The method of claim 12 wherein the composition is an aqueous gel material.

19. A method of formulating a composition for topical drug delivery comprising;
(a) heating water to about 90° C.,
(b) adding p-hydroxybenzoic acid methyl ester to the water and, while the water is allowed to cool, stirring at about 450 rpm,
(c) while the water is about 75° C. to about 80° C. adding hydroxyethylcellulose and carboxymethylcellulose and continue stirring at about 450 rpm,
(d) while the water is about 55° C. to about 60° C. adding methoxypolyethylene glycol and continue stirring at 450 rpm,
(e) dissolve a naturally occurring or synthetic prostaglandin in ethanol and adding the resulting ethanol solution to the aqueous solution with stirring at about 300 rpm, and
(f) adding propylene glycol to the aqueous solution and stirring at about 300 rpm and then reducing the stirring speed to about 90 rpm.

20. The method of claim 19 wherein the composition is formulated using a fixed torque mixer.

21. The method of claim 19 wherein the naturally occurring or synthetic prostaglandin is prostaglandin E1.

22. The method of claim 19 further comprising after adding the p-hydroxybenzoic acid methyl ester to the water the solution is stirred for about 2 hours.

23. The method of claim 19 further comprising after adding the hydroxyethylcellulose and carboxymethylcellulose the solution is stirred for from about 25 to about 40 minutes.

24. The method of claim 19 further comprising after adding the methoxypolyethylene glycol the solution is stirred for about 6 hours.

25. The method of claim 19 further comprising after adding the naturally occurring or synthetic prostaglandin dissolved in ethanol to the aqueous solution the solution is stirred for about one hour.

26. The method of claim 19 further comprising after adding the propylene glycol to the aqueous solution the solution is stirred at about 300 rpm for about one hour and then stirring at about 90 rpm for from about 12 hours to about 18 hours.

27. The method of claim 19 further comprising packaging the composition in a tube.

28. The method of claim 27 wherein the tube is made of polypropylene.

29. The method of claim 27 wherein the tube contains a single dose of the composition.

30. The method of claim 27 further comprising freezing the composition in the tube.

31. The method of claim 19 wherein the composition is an aqueous gel material.
